(19) United States
(12) Patent Application Publication
(10) Pub. No.: US 2021/0177761 A1
(43) Pub. Date: Jun. 17, 2021

(54) MUCOADHESIVE POLYMER COMPOSITIONS

(71) Applicant: Joseph Domenic SCIAMANNA, Elkton, MD (US)

(72) Inventor: Joseph Domenic SCIAMANNA, Elkton, MD (US)

(21) Appl. No.: 17/113,246

(22) Filed: Dec. 7, 2020

Related U.S. Application Data
(60) Provisional application No. 62/948,559, filed on Dec. 16, 2019.

(51) Int. Cl. A61K 9/16; C08L 5/04; A61K 47/36

(52) U.S. Cl. CPC A61K 9/1652; C08L 5/04; A61K 47/36; C08L 2205/18; C08L 2203/02; C08L 2205/03; C08L 2201/06; C08L 2201/54; C08L 2205/025

(57) ABSTRACT

Compositions of polymer matrices inclusive of hydrophobically modified and pH-responsive polymers which adhere to a biological tissue for use in delivery of active agents to mammalian subjects are provided.

This patent application claims the benefit of priority from U.S. Provisional Application Ser. No. 62/948,559, filed Dec. 16, 2019, teachings of which are herein incorporated by reference in their entirety.

### FIELD

This disclosure relates to compositions of polymer matrices inclusive of hydrophobically modified and pH-responsive polymers which adhere to a biological tissue. The compositions are useful for delivery of active agents to mammalian subjects.

### BACKGROUND

Modern advancements in biotechnology have provided a plethora of polypeptides and macromolecules capable of affecting physiological changes in mammals.

However, there are many contributing factors which can affect the oral bio-availability of drugs in the gastrointestinal tract including thickness of the epithelium, the surface area, blood flow, local physical and chemical environment and characteristics of the drug substance itself, including its solubility in water, its chemical stability, molecular weight and particle size. Further, before a bioactive compound is transferred from the intestinal lumen to the blood, the compound may be subject to degradation or deactivation by the various components of the lumen.

Accordingly, while these new active agents exist, many are unable to enter the bloodstream through an oral or non-invasive route.

Scientists have developed a variety of methods of drug delivery through the encapsulation of these active agents in various forms.

Biodegradable particles have been developed as sustained release vehicles used in the administration of small molecule drugs as well as protein, peptide and nucleic acid drugs. These particles are typically encapsulated in a polymer matrix which is biodegradable and biocompatible. As the polymer is degraded or dissolved and/or as the drug diffuses out of the polymer, the drug is released into the body. Polymers oftentimes used in preparing these particles are polyesters such as poly(glycolide-co-lactide) (PLGA), polyglycolic acid, poly-β-hydroxybutyrate, and polyacrylic acid ester. In addition to providing for sustained release, these particles have the additional advantage of protecting the drug from degradation by the body.

Oral route is the most commonly used and preferred route of administration for active agents. However, bio-availability remains a challenge for many therapeutic agents and can result in undesirable variations in the rate and extent of absorption.

Water-soluble drugs, and in particular protein-based physiologically active agents, have low stability in the gastrointestinal tract and low permeability through the wall of intestinal tract. Thus intravenous injection is oftentimes used for these drugs. At the same time, poorly soluble agents are oftentimes entrapped in lipids or oil emulsions and have limitations of their rapid clearance from circulation due to uptake by reticulo-endothelial system (RES), primarily in the liver.

Various techniques including liposomal preparations, lipid polymer bilayer formation, use of bio-erodible mucoadhesive polymers to avoid first pass metabolism, coating to avoid peptic degradation and formation of nanoparticles have been suggested.

EP2403447B1 discloses an oral polymeric delivery vehicle for transmucosal delivery of a bioactive agent in a mammalian subject. The oral formulation inhibits degradation of the bioactive compound within the stomach and within the lumen of the intestine by encapsulation within a polymeric shell which prevents dissolution until after passing through the mucosal wall of the small and/or large intestine. Thus, enzymatic degradation of the delivery vehicle containing the bioactive compound is substantially inhibited until after absorption of the delivery vehicle into blood vessels of the intestinal mucosa. However, this delivery vehicle is limited in its complication and requirement for covalent conjugation.

Australian Patent Application 2004/305395 B2 discloses orally administrable nanoparticle compositions having an enhanced entrapping rate of water-soluble drugs within nanoparticles composed of lipids and polymers suggested to be stable against lipases. The nanoparticles are prepared by binding water-soluble drugs with counter-ion substances and adding lipids, polymers, and emulsifiers thereto. However, these nanoparticles are relatively unstable and have slow drug release.

Available systems are somewhat effective in delivering poorly bio-available drugs through the mucosal membrane after oral delivery, but have drawbacks that prevent their widespread use, including poor transport through the mucosal membrane or excessive cost, insufficient drug entrapping rate and low stability.

Traditional alginate microbeads can protect encapsulated agents from the acidic conditions of the stomach and releases the target compounds in the in less acidic conditions of the small intestine. However, traditional alginate matrices do not provide any mucoadhesive properties to the microbeads.

There is a need for drug delivery formulations with enhanced mucoadhesivity which provide for absorption of poorly permeable drugs across tissues such as the intestinal epithelium.

### SUMMARY

An aspect of the present invention relates to compositions comprising a polymer matrix capable of adhering to a biological tissue. The polymer matrix comprises a hydrophobically modified polymer and a pH-responsive polymer. In one nonlimiting embodiment, the polymer matrix is extruded into beads or spheres.

Another aspect of the present invention relates to a composition for delivery of an active agent. The composition comprises a polymer matrix comprising a hydrophobically modified polymer and a pH-responsive polymer and an active agent loaded on the polymer matrix.

In one nonlimiting embodiment, the polymer matrix is extruded into beads or spheres which are then loaded with active agent via mixing or diffusion.

Another aspect of the present invention relates to a method for producing a composition for delivery of an active agent. The method comprises mixing or diffusing the active agent into a polymer matrix comprising a hydrophobically modified polymer and a pH-responsive polymer.

In one nonlimiting embodiment, the polymer matrix is extruded into beads or spheres which are then loaded with active agent via mixing or diffusion.

Yet another aspect of the present invention relates to a method for delivering an active agent to a mammalian subject. The method comprises administering to the mammalian subject a composition comprising an active agent loaded onto polymer matrix comprising a hydrophobically modified polymer and a pH-responsive polymer.

### DETAILED DESCRIPTION

Provided by this disclosure is a biocompatible polymer delivery composition for active agents. The polymer delivery system is particularly useful for delivery of active agents such as peptides, lipids, macromolecules and other biological agents which, when administered without encapsulation, would be denatured by the digestive process. Further, unlike many alternative polymer matrices, the biocompatible polymer delivery composition of this disclosure is able to achieve mucoadhesivity without sacrificing pH responsiveness.

Compositions of this disclosure comprise a polymer matrix formed of at least one hydrophobically modified polymer and at least one pH-responsive polymer.

Nonlimiting examples of pH-responsive polymers which can be used in these compositions include alginic acids such as sodium alginate, potassium alginate, magnesium alginate, calcium alginate, and aluminum alginate.

Nonlimiting examples of polymers for hydrophobic modification which can be used in these compositions include but are not limited to: chitosan, including chitosan lactate, chitosan salicylate, chitosan pyrrolidone carboxylate, chitosan itacanate, chitosanniacinate, chitosan formate, chitosan acetate, chitosan gallate, chitosan glutamate, chitosan maleate, chitosan aspartate, chitosan glycolate and quaternary amine substituted chitosan and salts thereof. Chitin can also be used.

In one nonlimiting embodiment, only a portion of the polymer is hydrophobically modified so that the polymer matrix comprises a chitosan/HP-MC polymer mix. In one nonlimiting embodiment, 0.1%-50% of the polymer is hydrophobically modified.

In another nonlimiting embodiment, all of the polymer is hydrophobically modified.

In one nonlimiting embodiment, the polymer matrix comprises a layer of hydrophobically-modified alginate.

A single hydrophobic substituent or a plurality of hydrophobic substituents can be attached to the polymer to produce the hydrophobically modified polymer. The percent by weight of the substituent or substituents can vary depending on desired use. Nonlimiting examples of hydrophobic substituent include: hydrophobically modified chitosan; compounds having an alkyl, aryl or arylalkyl group; compounds having a carbonyl functional group such as aldehydes, ketones, carboxylic acids, esters, amides, enones, acyl chlorides, acid anhydrides, urea and carbamates; electrophiles; and any active organic compound capable of forming a Schiff base with a nucleophilic partner during the reaction.

In one nonlimiting embodiment a plurality of hydrophobic substituents having a backbone of carbon atoms of at least six or more carbon atoms, more preferably six to no more than thirty-six carbon atoms, more preferably ten to twenty carbon atoms, and more preferably C12H26, is used to affect the chemical or physical properties of the polymer.

For hydrophobic substituents including at least one side chain, the side chain may be any type of organic or inorganic hydrophobic group or compound.

In some nonlimiting embodiments, the hydrophobic substituent undergoes a chemical reaction process before or after the substituent is added to the polymer such as an addition reaction process, an addition reaction process followed by an elimination reaction process (addition-elimination process), a nucleophilic addition reaction process, a 1,2 nucleophilic addition reaction process, a nucleophilic acyl substitution reaction process, or an alkylimino-de-oxo-bi-substitution reaction process.

Without being bound to any particular theory, it is believed that hydrophobic modification of the polymer enhances mucoadhesivity of the polymer matrix as compared to polymer matrices of similar polymers without hydrophobic modification.

As will be understood by the skilled artisan upon reading this disclosure, the molecular weight and the degree of cross-linking of the polymers in this matrix can be adjusted to control the decomposition rate of the polymer and thus the release rate of any active agent. Methods of controlling molecular weight and cross-linking to adjust release rates are well known to those skilled in the art.

In one nonlimiting embodiment, the polymer matrix is cross-linked in calcium or another comparable divalent cross-linking solution.

The polymer matrix may further include at least one reagent, catalyst, excipient, transporter, penetrating agent, stabilizer, modifier, foaming agent, or plasticizer. The polymer matrix may further include a functional group such as a Lewis base, Bronsted base, a nucleophile, any active organic compound capable of forming a Schiff base with an electrophilic partner during the reaction, or any active organic compound that is capable of donating a proton to an electrophilic partner during the reaction process.

Further, the polymer matrix may further include cellulosics such as, but not limited to, hydroxyethyl cellulose, hydroxypropyl cellulose, methyl cellulose, hydroxypropyl methyl cellulose, or hydroxyethyl methyl cellulose. In one nonlimiting embodiment, the formed polymer matrix may include chitosan or chitosan derivatives, alginate, and cellulosic, including any combinations or mixtures of the chitosan, alginate and the cellulosic.

In one nonlimiting embodiment, the polymer matrix is prepared from an aqueous-based solution of the hydrophobically modified polymer and pH-responsive polymer. In one nonlimiting embodiment, this aqueous solution is extruded into beads or spheres. In one nonlimiting embodiment, the beads range in size from between 0.5 mm and 3 mm in diameter. The beads comprise between 0.1 and 5% pH-responsive polymer and between 0.1 and 3% by weight of a hydrophobically-modified polymer.

As will be understood by the skilled artisan upon reading this disclosure, alternative shapes for the polymer matrix such as, but not limited to films, stents, rings and rods can also be used.

The polymer matrix may be formed into solid state ranging from a solid to a sponge-like texture; or it may exist in a liquid state when administered with at least one polymer with both hydrophobic and pH-responsive properties which hardens in acidic pH ranges occurring in the stomach.

Properties exhibited by the polymer matrices of this disclosure include low toxicity, hydrophilicity, biocompatibility, biodegradation, acid catalysis, polyelectrolytic, self-assembling component factor, aromaticity, pH sensitivity, solubility in water and acidic to neutral solution, anti-bacterial, covalent bonding, amphipathic compound attractor and wherein the polymeric substrate includes at least one physical property of macromolecular in size, bearing hydrophobic groups, polymerization, aliphatic linear hydrocarbon compound, scaffolding or lattice matrix structure, aromaticity, delocalized electrons, flexibility, adhesiveness, transparency, opaqueness, stoichiometric conformations similar to D-glucosamine sugars, gelatinous factor, viscosity factor, angular frequency, shear rate, light scattering, and color hues.

Once the delivery composition is formed into the desired viscosity and compositional formula, it is then administered. In one nonlimiting embodiment, the composition is administered orally. However, as will be understood by the skilled artisan upon reading this disclosure, alternative routes of administration may be used.

Once administered, the polymer matrix with hydrophobic substituent is capable of adhering to a desired living tissue. For instance, it may be desirable to have the composition adhere to the intestinal lining and gradually pass through the lining into the circulatory system, where it gradually releases the active agent at a rate determined by the decomposition rate of the matrix. Alternatively, the polymer delivery composition can target damaged tissue or red blood cells where the hydrophobic substituent adheres to desired tissue.

In one nonlimiting embodiment, the active agent is loaded into beads or spheres extruded from the polymer matrix via diffusion in an aqueous solution where both the solid beads or spheres and the active agent are stable. For alginate beads or spheres and active peptide agents, a loading solution that ranges from pH 5-pH 7 and 2-10° C. is used to ensure bead and peptide integrity. The diffusion process can be enhanced by continuous stirring over the duration of the diffusion. When the mucoadhesive polymer comprises chitosan, a preferred active agent is a molecule with lightly negative charge (pKa >7.1) to associate with the positively charged chitosan. The loading process takes approximately 30 minutes for complete loading in the case of complementary charged active agents and polymers. However, diffusion loading can be accomplished in reduced or increased times by modifying the volume of loading solution, the concentration of active agent, the concentration of polymer matrix, the stirring speed, duration of loading or the identities of the polymers or active agents.

The following nonlimiting examples are provided to further illustrate the present invention.

### EXAMPLES

### Example 1: Preparation of Polymer Matrix

A water-based solution was prepared from the below compounds for extrusion into beads at room temperature.

2% w/w alginic acid

0.25% w/w chitosan

0.2% w/w hydrophobically modified chitosan

The compounds were combined into a solution with DI water to form a solution. The resultant solution was then extruded as beads into a divalent cross-linking solution. The beads were washed and then placed in a glutaraldehyde or acid solution to cross link chitosan. Beads are then washed again and loaded with target molecule through diffusion or mixing.

### Example 2: Assessing Mucoadhesivity

A closed tube in which fluid is slowly passed through is lined with sections of rat intestine or a similar intestinal tissue. Microbeads of a polymer matrix of this disclosure are introduced to the fluid and allowed to adhere to the intestinal tissue. The pressure and flowrate of the fluid passing through the tube are incrementally increased to elute the microbeads from the tissue-lined tube. The quantity of microbeads eluted at each pressure and flowrate is recorded and compared to evaluate mucoadhesivity. To demonstrate enhanced mucoadhesivity of the polymer matrices of this disclosure with a hydrophobically modified polymer, comparisons can be performed with same weight compositions of a similar polymer matrix without hydrophobic modification.

What is claimed:

1. A polymer matrix capable of adhering to a biological tissue, said polymer matrix comprising a hydrophobically modified polymer and a pH-responsive polymer.

2. The polymer matrix of claim 1, wherein the hydrophobically modified polymer is hydrophobically modified by attachment of one or more hydrophobic substituents to the polymer.

3. The polymer matrix of claim 2, wherein the hydrophobic substituent has an alkyl, aryl, or arylalkyl group.

4. The polymer matrix of claim 1, wherein the pH-responsive polymer is an alginate.

5. The polymer matrix of claim 1, wherein the the hydrophobically modified polymer is a chitosan or chitin.

6. The polymer matrix of claim 5, wherein a portion of the chitosan or chitin is hydrophobically modified.

7. The polymer matrix of claim 1 further comprising hydroxyethyl cellulose, hydroxypropyl cellulose, methyl cellulose, hydroxypropyl methyl cellulose or hydroxyethyl methyl cellulose.

8. The polymer matrix of claim 1 further comprising an active agent.

9. The polymer matrix of claim 1 which exhibits enhanced mucoadhesivity as compared to a polymer matrix without a hydrophobically modified polymer.

10. A polymer bead or sphere comprising the polymer matrix of claim 1.

11. A composition for delivery of an active agent, said composition comprising a polymer matrix comprising a hydrophobically modified polymer and a pH-responsive polymer and an active agent loaded on the polymer matrix.

12. The composition of claim 11 which exhibits enhanced mucoadhesivity as compared to a polymer matrix without a hydrophobically modified polymer.

13. A method for producing a composition for delivery of an active agent, said method comprising mixing or diffusing an active agent into the polymer matrix of claim 1.

14. The method of claim 13 wherein the composition exhibits enhanced mucoadhesivity as compared to a composition comprising a polymer matrix without a hydrophobically modified polymer.

15. A method for delivering an active agent to a mammalian subject, said method comprising administering to the mammalian subject a composition comprising an active agent loaded onto polymer matrix comprising a hydrophobically modified polymer and a pH-responsive polymer.

16. The method of claim 15 wherein the composition exhibits enhanced mucoadhesivity as compared to a composition comprising a polymer matrix without a hydrophobically modified polymer.
